(19) United States
(12) Patent Application Publication
(10) Pub. No.: US 2017/0046586 A1
(43) Pub. Date: Feb. 16, 2017

(54) OPTICAL PROJECTION OVERLAY DEVICE

(71) Applicants: Adnan Abbas, Santa Barbara, CA (US); Melanie McWade, Nashville, TN (US); Anita Mahadevan-Jansen, Nashville, TN (US)

(72) Inventors: Adnan Abbas, Santa Barbara, CA (US); Melanie McWade, Nashville, TN (US); Anita Mahadevan-Jansen, Nashville, TN (US)

(21) Appl. No.: 14/822,447

(22) Filed: Aug. 10, 2015

(51) Int. Cl. G06K 9/32; A61B 5/00; H04N 9/31; G06K 9/20; G06T 7/00; H04N 5/33

(52) U.S. Cl. CPC G06K 9/3233; G06T 7/003; H04N 5/33; G06T 7/0014; H04N 9/3179; G06K 9/2027; G06K 9/2054; A61B 5/0075; G06T 2207/30004; A61B 2090/366

(57) ABSTRACT

Devices and methods for producing a highlighted overlay of a region, including an illumination source configured to illuminate the region at a bandwidth containing a first wavelength at a working distance, an imager at a working distance configured to image the illuminated region at a bandwidth containing a second wavelength, where the first and second bandwidths and wavelengths are not visible, a visible light projector configured to illuminate the region and registered to the imager to produce alignment of imaged feature with projected features at the same location on the region, and a controller executing a program configured to filter acquired images from the imager to identify areas of the region of a predetermined light intensity, and control the projector to visibly highlight those areas on the region.

The specification relates to illuminating a region that has areas emitting light at non-visible wavelengths and highlighting the emission areas of the region with a visible light overlay, and in particular for using the capability for surgical applications.

Fluorescence can be used to identify areas of a region including areas of surgical interest. Some materials may exhibit fluorescence at non-visible wavelengths. For these situations, which include some parts of the human body, detecting non-visible fluorescing areas and highlighting them visibly may be desirable.

### BRIEF DESCRIPTION

In some embodiments, devises and methods are provided that illuminate a region with light in a first wavelength range that is intended to excite emissions at a known non-visible wavelength range, which can be imaged with a suitable imager. All or part of the non-visible image scene may be projected visibly back onto the imaged region with visible light, which may be used to highlight areas of interest from the image.

In some embodiments a device for producing an overlay of a region may be provided, including an illumination source configured to illuminate the region at a bandwidth containing a first wavelength at a working distance, an imager at a working distance configured to image the illuminated region at a bandwidth containing a second wavelength, where the first and second bandwidths and wavelengths may not be visible, a visible light projector configured to illuminate the region and registered to the imager to produce alignment of imaged features with projected features at the same location on the region, and a controller executing a program configured to filter acquired images from the imager to identify areas of the region of a predetermined light intensity, and control the projector project a visible image of those areas on the region.

In some embodiments a method for producing an overlay of a region, may be provided including illuminating the region at a bandwidth containing a first wavelength at a working distance, imaging the illuminated region at a bandwidth containing a second wavelength, where the first and second bandwidths and wavelengths are not visible, filtering acquired images from the imager to identify areas of the region of a predetermined light intensity, and projecting a visible image of those areas on to the region with a projector aligned to the imager and the region.

In some embodiments the working distances may be greater than 10 cm from the region.

In some embodiments the working distances may be less than 100 cm from the region.

In some embodiments the working distances may be greater than 25 cm and less than 200 cm from the region.

In some embodiments the working distances may be both 50 cm ±10 cm.

In some embodiments the imager, illuminator, and projector may be configured as one unit at one working distance.

In some embodiments the illuminated, imaged and projected areas may be aligned and are less than 10 cm in the longest dimension.

In some embodiments the illumination wavelength may be substantially at 780-790 nm and may be filtered using standard optics to be narrowband.

In some embodiments the lightpath to the imager is bandpass filtered around 822 nm and the imager is capable of near infrared imagery.

In some embodiments the projected image is configured to be a single visible color, selected for high contrast with the colors of region of interest.

In some embodiments the filtering includes at least one of determining areas of the region within predetermined intensity levels, at predetermined variances from the average region intensity or near suspected locations of materials of interest.

### BRIEF DESCRIPTION OF THE DRAWINGS

FIG. 1 is an overview of overlay system used in particular medical application according to illustrative embodiments;

FIG. 2 is a block diagram of an illustrative device embodiment;

FIG. 3 is a flow chart of an illustrative method embodiment;

FIG. 4 is a block diagram of an illustrative parathyroid specific device embodiment:

### DETAILED DESCRIPTION OF ILLUSTRATIVE EMBODIMENTS

One or more embodiments described herein may provide for a visible highlighting of features of interest in a region determined from an images of those features acquired in a non-visible wavelength range.

One or more embodiments described herein may provide for visibly highlighting features of the human body for surgery.

One or more embodiments described herein may provide for visibly highlighting features for surgery by imaging areas of interest that fluoresce in a non-visible wavelength and projecting visible highlighting back onto those features.

One or more embodiments described herein may provide for visibly highlighting parathyroid regions of the thyroid by discriminating these areas due to variations in auto-fluorescence behavior and projecting visible highlights onto the parathyroid regions to aid in surgery.

Various aspects of the embodiments may include any combination of processing elements that may include computing devices executing software routines, such devices including computers and Personal Electronic Devices, as well as programmable electronics, logic circuits and other electronic implementations. Various combinations of optical elements may be employed including lasers, LED's and other light sources, filters, lenses, mirrors, beamsplitters and the like. The details of the optical, electronic, and processing embodiments described herein are illustrative and are not intended as limiting as alternative approaches using other combinations of like elements may be used to accomplish the same results in essentially the same manner

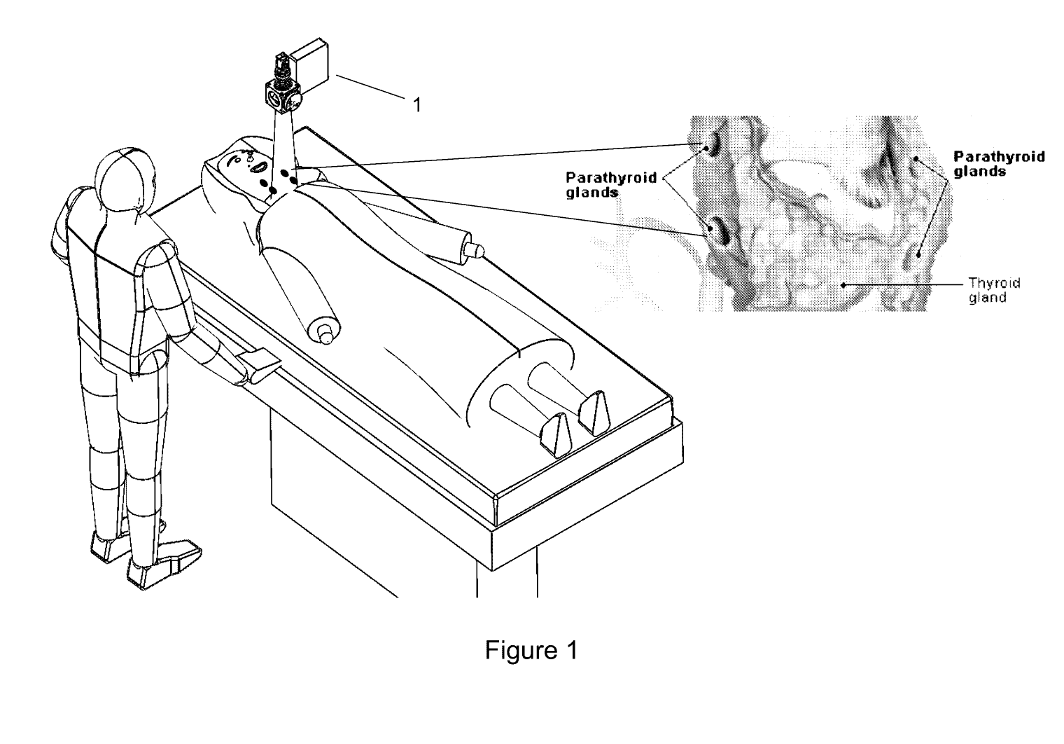

A method for discriminating parathyroid material from thyroid material, using auto-fluorescence, is described in U.S. patent application Ser. No. 13/065,469. This application has inventors in common with the current application, and is incorporated by reference in its entirety. This application discloses that when exposed to radiation in a narrow band about 785 nm, which is just outside visible range, both the thyroid and the parathyroid auto-fluoresce in a wavelength range above 800 nm, also not visible, sometimes centered at 822 nm, and that the parathyroid regions fluorescence intensity is significantly higher than the thyroid regions. This effect can be used to discriminate between the two areas for surgery, for even though the general locations of parathyroid tissue are known, they are hard to discriminate visually accurately enough for surgery, which can be a problem with parathyroid surgery. One detailed embodiment disclosed herein may be applicable to parathyroid surgery. As shown in FIG. 1, an optical overlay device 1 according to an embodiment of the current disclosure may be used to visibly highlight the parathyroid regions of a patient's exposed internal neck region during surgery.

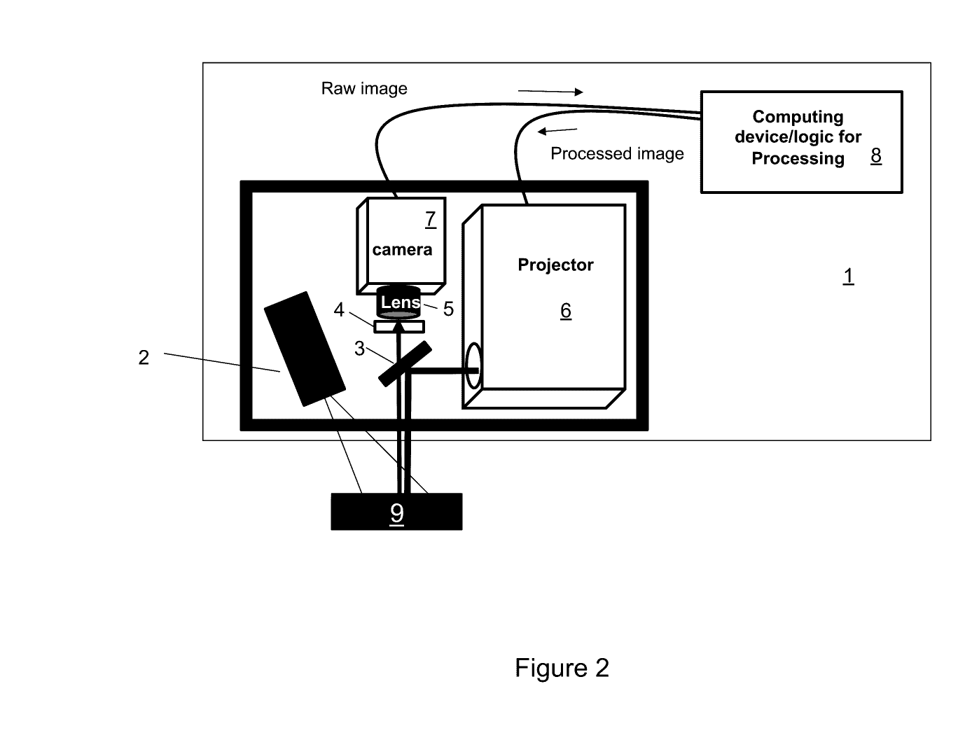

FIG. 2 shows an illustrative device embodiment for the overlay projector 1. Light source 2 at a first wavelength bandwidth illuminates a region of interest 9. The first wavelength bandwidth is used to stimulate emissions or fluorescence at a second wavelength bandwidth, which may be different from the first, expected from areas to be indentified of the region of interest. Those emission wavelengths are not visible for applications utilizing the device. An optional filter 4 may be used to pass wavelengths within the emission bandwidth and block others. An optional lens may be used 5 to set working distance of the device. Camera 7 is chosen to be capable of imaging the emission wavelength bandwidth or at least the portion passed through the filter 4. The image is acquired by computing device/logic 8 which also controls a visible light projector 6. Controllable Projector 6 and camera 7 are registered such that the imaged area and the projected area are aligned both in orientation and size so that features in the camera image or any portion of the camera image project back down on the region 9 such that their visible projection aligns precisely onto the actual physical features. The registration may be accomplished through optical design, which may be improved using calibration regions with definable edges and programming the projector to match such calibration pieces at a desired working distance for actual operation. Such edge or other feature detection may be updated in actual use by observing and correlating image features in actual regions of interest. The projection may be made co-linear with the imaging axis by use of a partially reflective element 3, such as a beamsplitter. Obviously different optical arrangements, such as which elements are on or off axis, may be accomplished with different arrangements of optical elements ands still function as described for the illustrative arrangement of FIG. 2.

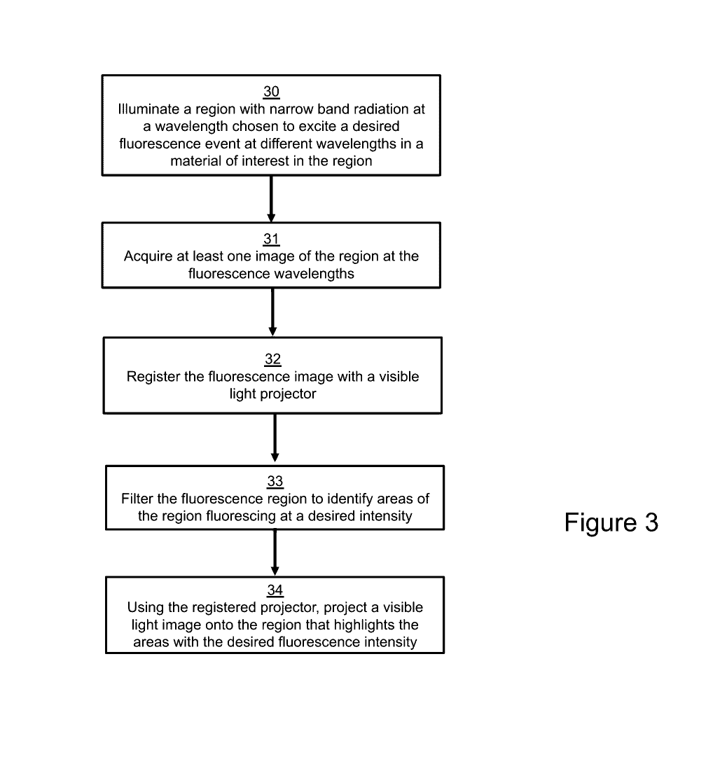

FIG. 3 is a flow chart of a method of operating a device such as the one shown in FIG. 2 for a case where a non-visible fluorescence of a material of interest may be used to identify the locations of that material in a region. In step 30 a region is illuminated with light, which may be narrowband, at a wavelength chosen to excite a desired non-visible fluorescence of a material of interest. In step 31 one or more images are acquired in a bandwidth that includes the fluorescence wavelength band. In step 32 the fluorescence image is registered with a visible light projector. This step may be performed or updated on an ongoing basis or just at the initial set-up of the device. In step 33 the image is filtered to select locations with a predetermined desired intensity at the fluorescence wavelength. Filtering may include, high/low intensity, deviation from average, within a range, near predetermined locations, or any combination thereof, or other filtering/processing techniques. The desired result is identifying locations likely to be the material of interest. In step 34, the projector is controlled to project all or part of the acquired image, which may be just of the selected locations, back onto the region with visible light. This will have the effect of illuminating the selected locations on the region with visible highlighting.

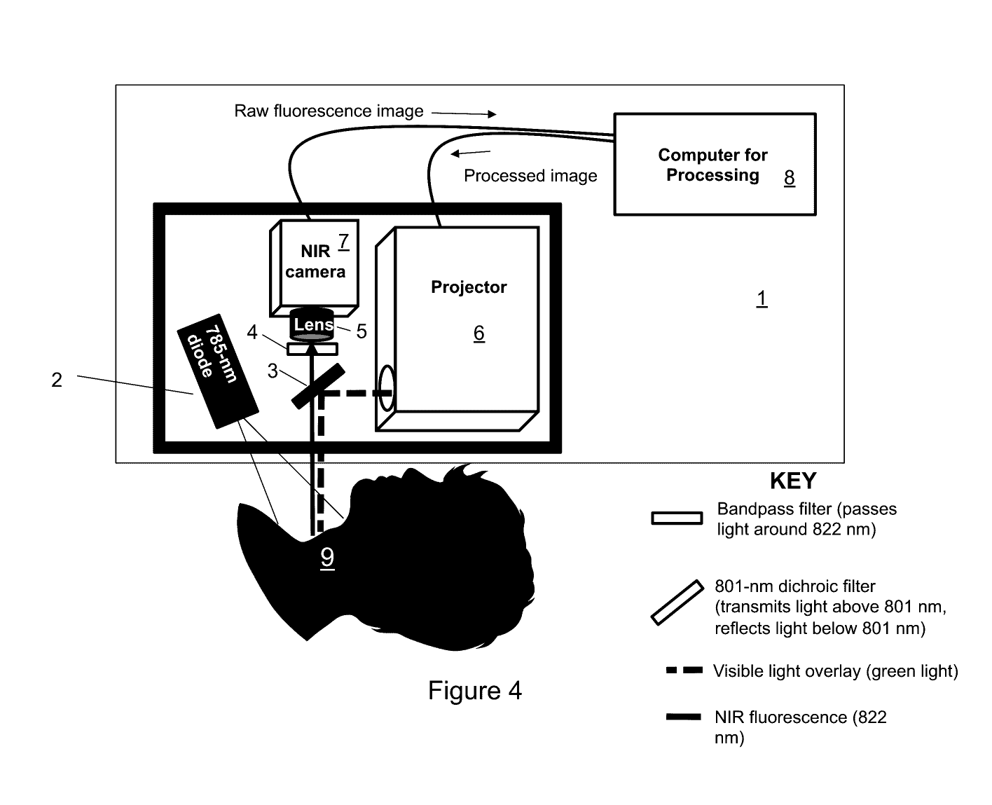

FIG. 4 shows a device 1 such as that of FIG. 2 configured for use in detecting parathyroid locations in a region 9, which is the frontal neck area of a patient opened for surgery. Illuminator 2 may be a 785 nm diode laser or equivalent source, preferably with a bandwidth less than 10 nm and a power of at least 10 mW, positioned at a working distance chosen conveniently for surgery. If Device 1 is mounted vertically, the distance is preferably between, 10 and 200 cm, more preferably between 25 and 100 cm above the neck, and in tested embodiment at 50 cm ±10 cm. Illuminator 2 is shown illuminating at an angle, but the illumination could be on the same axis as the imaging axis by use of another partially transmissive element, not shown. The 785 nm illumination is known to stimulate auto-fluorescence of the parathyroid around 822 nm. Thus filter 4, which passes radiation around 822 nm, preferably with a cut-off higher than 790 but lower than 830 nm, is employed in front of lens 5, which used to set the working distance which is preferably set the same as the illumination distance for convenient packaging of the device 1 into one or more co-located units. The 822 nm image is imaged by a near IR imager 7. That image is acquired by computer (and/or other processing logic) 8, which determines locations of the image whose intensities in this imaged wavelengths meet predetermined criteria identifying these as locations of the parathyroid areas. In many cases these may be the highest intensity regions identified, or may be located as discreet relatively bright areas near the parathyroid suspected locations, which as described in the incorporated reference are generally known, just not accurately enough for surgery. The processor determines the likely locations of the parathyroid areas based on the image and controls registered projector to project back onto the opened neck those selected areas in visible light, in this case green light through partially transmissive element 3. Since visibly an opened neck area is mostly reddish and brownish, green is a high contrast highlight color. The result is as shown in FIG. 1 where the parathyroid areas are accurately, visibly highlighted. If bright limited-area features are detected near the known locations of the parathyroid tissue, they are highly likely to be parathyroid tissue. If no bright areas are detected near the appropriate locations, the surgeon may default to other, possibly less convenient, location techniques

The embodiments described herein are exemplary. Modifications, rearrangements, substitute devices, processes etc. may be made to these embodiments and still be encompassed within the teachings set forth herein.

1. A device for producing an overlay of a region, comprising;
an illumination source configured to illuminate the region at a bandwidth containing a first wavelength at a working distance;
an imager at a working distance configured to image the illuminated region at a bandwidth containing a second wavelength, where the first and second bandwidths and wavelengths are not visible;
a visible light projector configured to illuminate the region and registered to the imager to produce alignment of imaged feature with projected features at the same location on the region, and;
at least one of a controller executing a program or logic configured to filter acquired images from the imager to identify areas of the region of a predetermined light intensity, and control the projector to project a visible image of those areas those areas on the region.

2. The system of claim 1 wherein the working distances are greater than 10 cm from the region.

3. The system of claim 2 wherein the working distances are less than 100 cm from the region.

4. The system of claim 2 wherein the working distances are greater than 25 cm and less than 200 cm from the region.

5. The system of claim 1 wherein the working distances are both 50 cm ±10 cm.

6. The system of claim 1 wherein the imager, illuminator, and projector are configured as one unit at one working distance.

7. The system of claim 1 where the illuminated, imaged and projected areas are aligned and are less than 10 cm in the longest dimension.

8. The system of claim 1 wherein the illumination wavelength is substantially at 780-790 nm and is filtered using standard optics to be narrowband.

9. The system of claim 1 wherein the lightpath to the imager is highpass filtered above 790-830 nm and the imager is capable of near infrared imagery.

10. The system of claim 1 wherein the projected image is configured to be a single visible color, selected for high contrast with the colors of region of interest.

11. The system of claim 1 wherein the filtering includes at least one of determining areas of the region of within predetermined intensity levels or at predetermined variances from the average region intensity.

12. A method for producing an overlay of a region, comprising;
illuminating the region at a bandwidth containing a first wavelength at a working distance;
imaging the illuminated region at a bandwidth containing a second wavelength, where the first and second bandwidths and wavelengths are not visible;
filtering acquired images from the imager to identify areas of the region of a predetermined light intensity, and;
projecting a visible image of the identified areas on to the region with a projector aligned to the imager and the region.

13. The method of claim 12 wherein the working distances are greater than 10 cm from the region.

14. The system of claim 13 wherein the working distances are less than 100 cm from the region.

15. The method of claim 13 wherein the working distances are greater than 25 cm and less than 200 cm.

16. The method of claim 12 wherein the working distances are both 50 cm ±10 cm.

17. The method of claim 12 wherein the imager, illuminator, and projector are configured as one unit at one working distance.

18. The method of claim 12 where the illuminated, imaged and projected areas are aligned and are less than 10 cm in the longest dimension

19. The method of claim 12 wherein the illumination wavelength is substantially at 780 to 790 nm and is filtered using standard optics to be narrowband.

20. The method of claim 12 wherein the lightpath to the imager is highpass filtered around 822 nm and within 790-830 nm and the imager is a near infrared imager.

21. The method of claim 12 wherein the projected image is configured to be a single visible color, selected for high contrast with the colors of region of interest.
